(19) United States
(12) Patent Application Publication
(10) Pub. No.: US 2026/0127561 A1
(43) Pub. Date: May 7, 2026

(54) METHODS AND SYSTEMS TO INDUCE REFLECTION BASED ON EMOTIONAL VALENCE

(71) Applicant: Sequelae, Inc., Minneapolis, MN (US)

(72) Inventors: Sara Taylor, Edina, MN (US); Natalie Sheils, St. Paul, MN (US); Danielle Marria Martin, Minneapolis, MN (US); Scott Archibald, Wayzata, MN (US); Duncan Hieu Minh Dam, Palm Springs, CA (US); Salomea Anna Jankovic, Minneapolis, MN (US); Alex Ruch, Prosper, TX (US); Steven J. Catani, Athens, GA (US)

(73) Assignee: Sequelae, Inc., Minneapolis, MN (US)

(21) Appl. No.: 19/354,420

(22) Filed: Oct. 9, 2025

Related U.S. Application Data
(60) Provisional application No. 63/705,633, filed on Oct. 10, 2024.

(51) Int. Cl. G06Q 10/40; G06F 40/20

(52) U.S. Cl. CPC G06Q 10/40; G06F 40/20

(57) ABSTRACT

Methods and systems to induce reflection based on emotional valence are provided. A digital communications network (DCN) is provided to a plurality of members of a population to communication with each other. A first communication is transmitted to an individual member of the plurality of members and a first response is received from the individual member. The first response is analyzed to determine a first level of emotional valence and a second communication is generated and designed to induce reflection in the individual member for a first time. The second communication is transmitted to the individual member and a second response is received. The second response is analyzed to determine a second level of emotional valence.

### CROSS REFERENCE TO RELATED APPLICATIONS

This application claims the benefit of U.S. Provisional Application No. 63/705,633, filed on Oct. 10, 2024, which application is incorporated herein by reference in its entirety.

### BACKGROUND

Various embodiments relate generally to inducing reflection in an individual member of a population an improving an emotional valence of the individual member via a series of communications to the individual member and responses from the individual member.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

People often take actions - whether simple, hard, good, or bad, etc. - that impact their health. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences; thus, it is desirable for people to reflect on their actions and potential consequences.

Mindfulness, as gained through reflection, is an essential part of self-improvement. Unless someone is aware of their behaviors and how those behaviors impact the things that are important to them, it is very hard to sustain change in their behaviors. Reflection is the process by which people gain that understanding. Reflecting takes conscience effort and is often emotionally draining and cognitively draining. Thus, a person may find it easier to not reflect on such actions.

Computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities.

What is needed is a way to induce reflection in an individual member of a population and improving an emotional valence of the individual member via a series of communications to the individual member and responses from the individual member.

### SUMMARY

Example aspects of the present disclosure include:

A method to induce reflection according to at least one embodiment of the present disclosure comprises providing a digital communications network (DCN) for a plurality of members of a population to communicate with each other; transmitting a first communication by the DCN to an individual member of the plurality of members; receiving a first response to the first communication from the individual member; analyzing the first response to determine a first level of emotional valence of the individual member; generating a second communication by the DCN designed to induce reflection in the individual member for a first time to increase the first level of emotional valence; transmitting the second communication to the individual member; receiving a second response to the second communication from the individual member; and analyzing the second response to determine a second level of emotional valence of the individual member.

Any of the aspects herein, further comprising: generating a third communication by the DCN designed to induce reflection for a second time in the individual member to increase the first level of emotional valence when the second level of emotional valence is less than the first level of emotional valence.

Any of the aspects herein, further comprising: generating a third communication by the DCN designed to induce reflection for a second time in the individual member to increase the first level of emotional valence when a difference between the first level of emotional valence and the second level of emotional valence is less than an emotional valence threshold.

Any of the aspects herein, further comprising: transmitting a request to the individual member to share their reflection in the DCN when a difference between the first level of emotional valence and the second level of emotional valence is greater than an emotional valence threshold.

Any of the aspects herein, further comprising: analyzing at least one of: one or more communications in the DCN by the individual member or biometric test results provided to the DCN by the individual member to generate the first communication and the second communication.

Any of the aspects herein, wherein the first communication and the second communication comprise a first question and a second question, respectively.

Any of the aspects herein, wherein transmitting the first communication includes transmitting an incentive with the first communication.

Any of the aspects herein, wherein the induced reflection in the second communication is designed to induce reflection by the individual member about an action the individual member took.

Any of the aspects herein, wherein the induced reflection in the third communication is designed to induce reflection by the individual member about a cause of the difference between the first level of emotional valence and the second level of emotional valence.

Any of the aspects herein, wherein the induced reflection in the third communication is designed to induce reflection by the individual member about a cause of the second level of emotional valence being less than the first level of emotional valence.

A method to induce reflection according to at least one embodiment of the present disclosure comprises providing a digital communications network (DCN) for a plurality of members of a population to communicate with each other; transmitting a first communication to an individual member of the plurality of members; receiving a first response to the first communication; analyzing the first response to determine a first level of emotional valence of the individual member; generating a second communication designed to induce reflection in the individual member for a first time to increase the first level of emotional valence; transmitting the second communication to the individual member; receiving a second response to the second communication; analyzing the second response to determine a second level of emotional valence of the individual member; and generating a third communication designed to induce reflection for a second time in the individual member to increase the first level of emotional valence when a difference between the first level of emotional valence and the second level of emotional valence is less than an emotional valence threshold.

Any of the aspects herein, wherein the third communication by the DCN is generated when the second level of emotional valence is less than the first level of emotional valence.

Any of the aspects herein, further comprising: transmitting a request to the individual member to share their reflection in the DCN when a difference between the first level of emotional valence and the second level of emotional valence is greater than an emotional valence threshold.

Any of the aspects herein, further comprising: analyzing at least one of: one or more communications in the DCN by the individual member or biometric test results provided to the DCN by the individual member to generate the first communication and the second communication.

Any of the aspects herein, wherein the first communication and the second communication comprise a first question and a second question, respectively.

Any of the aspects herein, wherein transmitting the first communication includes transmitting an incentive with the first communication.

Any of the aspects herein, wherein the induced reflection in the second communication is designed to induce reflection by the individual member about an action the individual member took.

Any of the aspects herein, wherein the induced reflection in the third communication is designed to induce reflection by the individual member about a cause of the difference between the first level of emotional valence and the second level of emotional valence.

Any of the aspects herein, wherein the induced reflection in the third communication is designed to induce reflection by the individual member about a cause of the second level of emotional valence being less than the first level of emotional valence.

A system to induce reflection according to at least one embodiment of the present disclosure comprises a computer processor; a data repository in communication with the computer processor and storing: a plurality of communications including a first communication and a second communication; a plurality of responses including a first response and a second response; and a plurality of levels of emotional valence including a first level of emotional valence and a second level of emotional valence; a digital communications network (DCN) which, when executed by the computer processor, provides a network for a plurality of members of a population to interact with each other; and a server controller which, when executed by the computer processor: transmits the first communication from the DCN to an individual member of the plurality of members; receives the first response to the first communication from the individual member; analyzes the first response to determine the first level of emotional valence of the individual member; generates the second communication designed to induce reflection in the individual member for a first time to increase the first level of emotional valence; transmits the second communication to the individual member from the DCN; receives a second response to the second communication from the individual member; and analyzes the second response to determine the second level of the emotional valence.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Reflection is normally considered after an event. An individual member can observe a change and reflect on the cause of that change. This reflection helps an individual member understand why a change, good or bad, had occurred and can teach the individual member how to achieve better results. Often these changes are observed through objective data, such as, a biometric sample, results tracked through a smartwatch, or some other means that bring the change to life.

Reflections are an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

Reflection may be considered as part of the change process - a person does or observes something, and then reflects on the action or observation and the consequences. This process implies some degree of planning, either in adding reflection to a planned action, or scheduling periods to reflect (these can be formal as in professional counseling). Unfortunately, much of life is unplanned and unobserved.

A person can reflect on many aspects of their experience. For example, the topic of their reflection can help the person focus on specific elements. These elements can improve the reflection process, for example, by enabling the person to make a personal connection with the reflections, such as, associating the process with an inspirational quote, music, movie, etc.

The topic may also highlight parts of the person's life that may be impacted by the lifestyle condition. The topic may help the person identify changes in their daily routines, the time of day, the time of year and/or upcoming life events due to their lifestyle condition. Conversely, the person may reflect upon how the lifestyle condition may be influenced by their daily routines, the time of day, the time of year and/or upcoming life events.

Various embodiments overcome those problems, for example, by guiding a reflection based on an emotional valence of an individual member. By guiding the reflection and trying to improve the emotional valence of the individual member, the individual member reflecting on their lifestyle condition can better understand their situation and can be better positioned to handle their lifestyle condition.

The DCN can analyze the individual member's communications in the DCN in order to determine communications and/or questions to send to the individual member. This can help identify aspects that the individual member is passionate about and/or shows specific interest in and where emotional valence can be improved. The DCN may also base the communications and/or questions on various other factors, for example the individual member's phenotype, the lifestyle condition, biological test results, family history, etc.

The DCN may also time the reflection requests so as to improve the reflection. As one non-limiting example, the DCN may analyze posts about what the individual member is exercising to time the communications with the exercises. The DCN can then time sending a request for the member to reflect on their lifestyle condition at a point shortly after the individual member is expected to have completed an exercise.

The DCN may also follow-up on the communications by requesting additional reflection from the individual member. For example, the DCN may ask the individual member to reflect on the factors that caused their response to the automated message. The individual member may respond to the follow-up request which may lead to further communications. These communications may occur over a single session or multiple sessions. Multiple sessions may take place over a single day or several days (or weeks).

The individual member's responses may be provided in various ways, for example, textual, verbal, visual, or some combination thereof. The DCN may provide a tool for the member to provide the responses, such as a voice capture program to translate speech into text. The individual member may provide detailed impressions, indicate their expected results and/or indicate a level of comfort (or expected level of comfort) with the results.

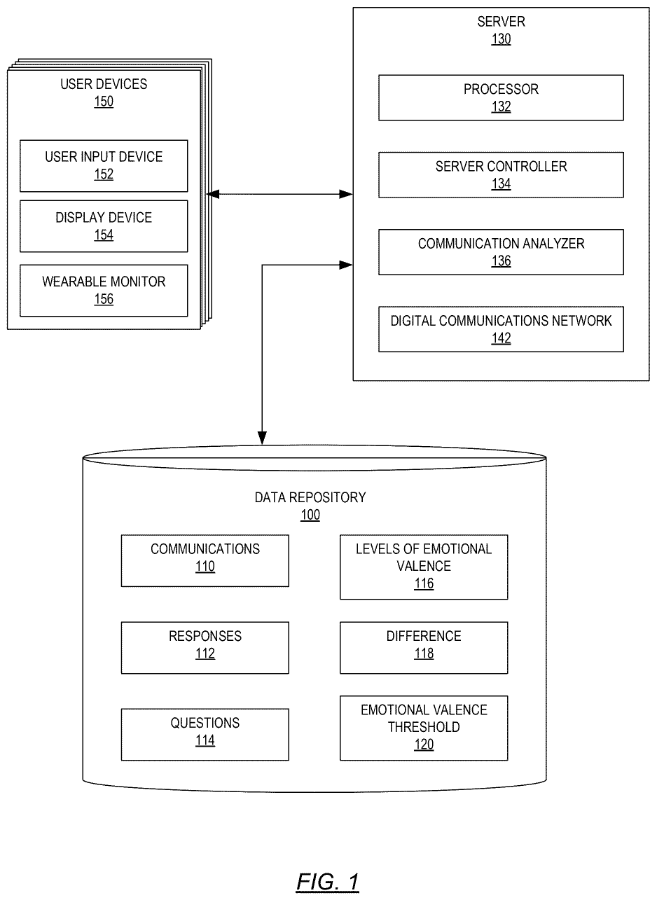

Turning to the Figures, the system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (110). The communications (110) are communications from members in a digital communication network (DCN) (142) (described in more detail below). The communications (110) may be, for example, text-based, image-based, multimedia communications, audio communications, or any combinations thereof. The communications may be between members, communications in a forum, communications with the DCN (142), or communications generated by the DCN (142) (e.g., communications from a conversation bot, also referred to as a chat bot).

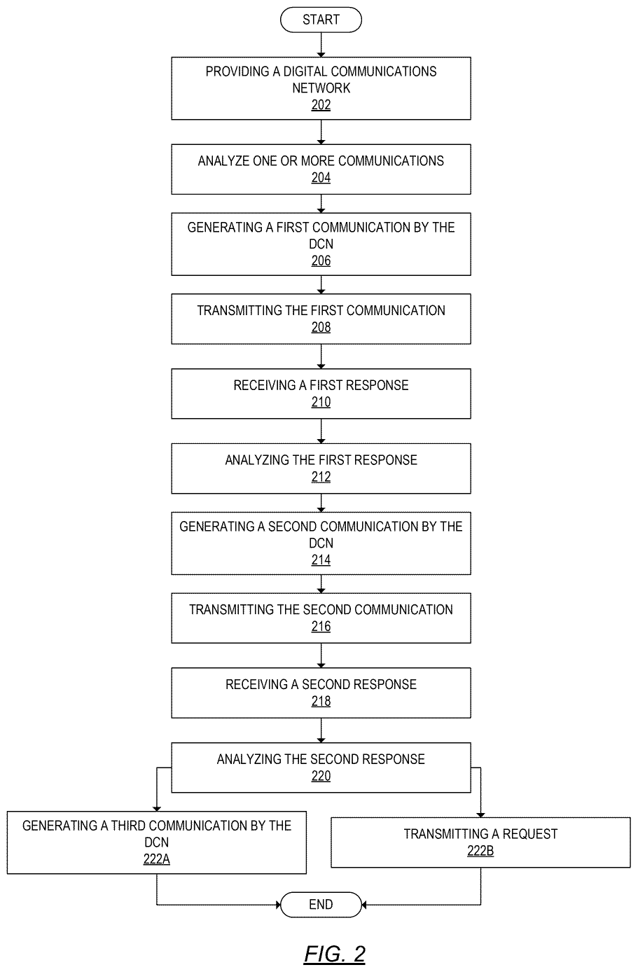

In some embodiments the communications (110) can include a first communication, a second communication, and/or a third communication generated by the DCN (142). In other embodiments, the communications (110) can include one communication, two communications, or more than two communications generated by the DCN (142). In some embodiments, the first communication, the second communication, and/or the third communication can be generated by the DCN (142) based on at least an analysis of communications (110) and/or responses (112) (described below) by the individual member. The communications (110) generated by the DCN (142) may be designed to induce reflection and improve an emotional valence in the individual member, as will be described in detail in the method of FIG. 2.

The data repository (100) also stores responses (112). The responses (112) are received from the individual member in response to the communications (110) generated and transmitted by the DCN (142). The responses (112) may include, for example, text and/or media (e.g., audio, visual, images, videos, etc.). The responses (112) can include an answer to a question in the communications (110) and/or reflections induced by the communications (110). The response (112) can be used to generate additional or follow up communications (110) from the DCN (142). The additional or follow up communications (110) may be used to, for example, induce further reflection in the individual member.

The data repository (100) also stores questions (114). The communications (110) generated by the DCN (142) can include questions (114) directed to the individual member and may include yes/no questions or open-ended questions. The questions (114) may be designed to induce a reflection in the individual member, understand a motivation of the individual member, collect information about the life and lifestyle of the individual member, etc. The questions (114) can include one question, two questions, or more than two questions. The questions (114) may also include a request for the individual member to answer the question (114). The request may include buttons (e.g., yes/no buttons), a text and/or media box for the individual member to type in or attach media.

The data repository (100) also stores levels of emotional valence (116). The levels of emotional valence (116) correspond to a positive degree or negative degree of emotion experienced by the individual member. The level of emotional valence (116) may change in response to different activities, communications (110), and/or a health of the individual member. For example, the individual member may have a low level of emotional valence (116) in response to news that the individual member has an adverse health condition. In another example, the individual member may have a high level of emotional valence (116) in response to participating in an activity such as jogging or yoga. The levels of emotional valence (116) can be determined in the responses (112) received from the individual member.

The data repository (100) also stores a difference (118) in the levels of emotional valence (116). In at least one embodiment, the difference (100) in the levels of emotional valence (116) is determined by subtracting a second or subsequent level of emotional valence (116) from a first or initial level of emotional valence (116). The difference (100) can be used to determine if the individual member's level of emotional valence (116) has increased or decreased. An increase in the individual member's level of emotional valence (116) may correspond to a positive increase and a decrease in the individual member's level of emotional valence (116) may correspond to a negative decrease in the individual member's level of emotional valence (116).

The data repository (100) also stores emotional valence thresholds (120). When the difference (118) meets or is greater than the emotional valence threshold (120), this may indicate a positive increase such that the individual member has moved from a negative emotional valence to a positive emotional valence. When the difference (118) is less than the emotional valence thresholds (120), this may indicate that the change in the levels of emotional valence (116) either decreased or the increase was not enough to indicate that the individual member has moved from a negative emotional valence to a positive emotional valence.

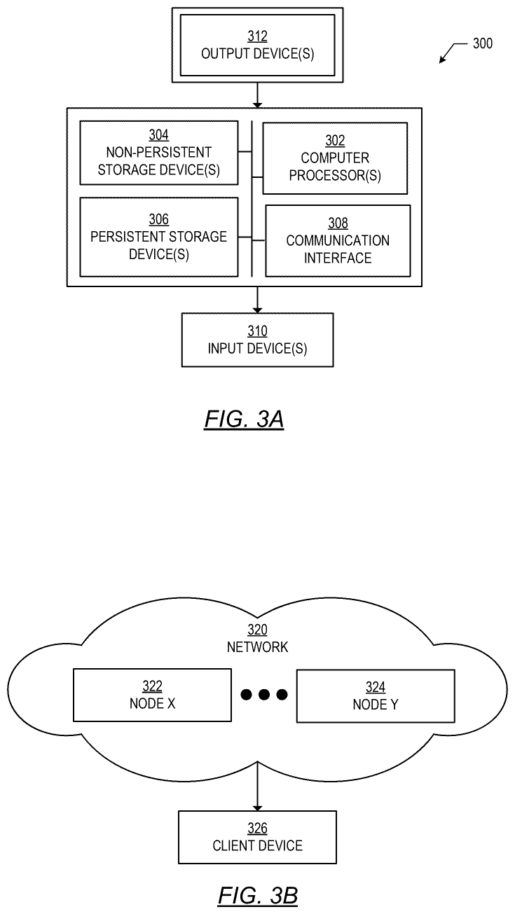

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (302) of FIG. 3A.

The server (130) also may include a server controller (134). The server controller (134) is software or application specific hardware which, when executed by the computer processor (136), controls and coordinates operation of the software or application specific hardware described herein. Thus, the server controller (134) may control and coordinate execution of the communication analyzer (136).

The server (130) also includes the communication analyzer (136). The communication analyzer (136) is software or application specific hardware which, when executed by the computer processor (132), analyzes communications (110) and/or responses (112) by the individual member and outputs communications (110) by the DCN (142). More specifically, the communication analyzer (136) may analyze communications (110) and/or responses (112) from the individual member, determine the level(s) of emotional valence (116) of the individual member, and output communications (110) (which may include, for example, the questions (114)) designed to induce reflection in the individual member to increase their level of emotional valence (116).

The server (130) also includes the DCN (142). The DCN (142) is a network through which members of a population can interact with each other, or with a system supported by the DCN (142). The DCN (142) can also interact with the members of the population and, for example, transmit communications (110) (which may include, for example, the questions (114)) to the individual member to induce reflection in the individual member.

In some embodiments, the DCN (142) may be, for example, an artificial intelligence bot configured to derive generated communications from analysis of existing communications (110) in the DCN (142) or biometrics provided to the DCN (142). Biometric data can be used in many instances such as the provisioning and commissioning of healthcare. For example, biometric data can be used to diagnose diabetes, prescribe hypertension medication with a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data's measurement, as well as the cost-benefit ratio of the intervention compared with other interventional options.

The biometric data, as well as reflections from the individual member, can be used in a “feedback” decision tree where, for example, an intervention can be prescribed or recommended to an individual member based on the biometric data and/or the reflections. Then the biometric data or additional reflections can be obtained to study the effects or results of the intervention.

In some embodiments, the conversation bot may use a visual or graphical interface, for example, a rendered avatar, or a plot or image from the bot or sharing one with the bot. The DCN (142) can send a visual representation showing that person's data from a wearable, their interaction with the community, their consistency in talking with the bot, etc. The conversation bot may then ask questions like “What do you notice” or “Is anything here surprising”in order to induce a reflection from the individual member.

The conversation bot can analyze data from various sources, in part, to formulate its communications with the individual member. These sources include data from i) the DCN (142), ii) the ongoing conversation with the individual member, and iii) previous conversations with the individual member. The conversation bot can also use temporal and situational cues to formulate its communication next with the member. When analyzing the data, the conversation bot may quote sections of the conversations for use with communications between the individual member and/or the conversation bot may identify patterns in the discussions. When formulating the communications, the conversation bot can analyze exchanges in the DCN (142) from other members. These communications can be limited to specifically target individuals, such as those deemed to be close contacts to the individual member or members that are considered influencers.

One-to-one communications with the conversation bot can be triggered and informed by one-to-many communications in the DCN (142), and conversations with the conversation bot can (a) refer individuals to the relevant communications in the DCN (142), (b) suggest posts to the DCN (142), and (c) make reference to ideas contained in posts. The two ways to interact can behave in a synergistic way and allow the conversation bot to include a “human in the loop”in a surprisingly scalable way.

During the communication between the individual member and the conversation bot, the DCN (142) can analyze the individual member's responses. Based on the input from the individual member, the DCN (142) may identify an adverse health condition or a lifestyle condition for the individual member. The conversation bot may then gather more information regarding the adverse health condition, for example, to develop more details to confirm the adverse health condition. The communication may be based on related experiences from another member of the population. The conversation bot may quote content from a post by that other member.

The synergy between the conversation bot and the DCN (142) can facilitate reflection on a topic. Alternatively, this can be seen to be reversed where the act of facilitating reflection synergizes with usage of the bot in the DCN (142).

Using analysis of the individual member's communications in the DCN (142), the conversation bot can suggest a reflection on a topic in a one-to-one communication. The suggestion may be made as part of a question to the individual member. The conversation bot may then focus its conversations with that individual member on that topic for a period of time (e.g., a number of exchanges, a few minutes, a few days, etc.). The conversation bot can incorporate excerpts of communication on the DCN (142) in its responses. The excerpts of communications may include post titles, post topics, quotes from the body of the communication, and/or post contributors. Also, the excerpts could be summaries of communications on the DCN (142). These summaries may be created automatically by the DCN (142) and/or are based on a topic, a member's communications, or a time period.

The bot may prompt the individual member to reflect on various classes of action. The action may be active or passive. The action may also be one that was contemplated even if not undertaken.

Two or more conversation bots, each with a different personality or a different role (e.g. facilitate reflections, find useful communications, say good morning and inspire a positive start to a day, handle routine network tasks, provide custom communication summaries), may be provided by the DCN (142). The family of bots could each use conversations between the other bots and the user on the DCN (142) to formulate their responses. When working with an individual member, the bots may be selected based on the personality of the bot that is expected to work best with that user. The decision may be based on past dealings with the individual member and/or through analysis of that individual member's communication on the DCN (142). For example, when attempting to induce a reflection, the bot may use a first personality, then switch to a second personality when helping draft a post regarding that reflection. Accordingly, various embodiments can be used to provide a method to improve the communications of the conversation bot through using authentic experiences of the individual member in the communication.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156). In an alternative embodiment, a separate wearable device may be in communication with the user device (150), such as a smart watch, or blood pressure monitor. The user devices (150) may also include a user input device (152) and/or a display device (154) through which the individual member can receive or send communications (110) including responses (112).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to induce reflection in an individual member of a population by determining and improving an emotional valence of the individual member via a series of communications to the individual member and responses from the individual member. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 202, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots.

At Block 204, a step of analyzing one or more communications in the DCN is provided. The communications may be the same as or similar to the communications (110). The communication may be analyzed by, for example, a communication analyzer such as the communication analyzer (136).

At Block 206, a step of generating a first communication by the DCN is provided. The first communication made be generated based at least on the analysis of the individual member's communications in the DCN or with the DCN. For example, communications by the individual member may indicate that the individual member has taken up jogging or yoga to improve their adverse health condition. Such information can be determined through searching or key-word searching the individual member's communications. The first communication can also be generated based on other information obtained from the individual member such as, for example, activity information obtained from a wearable device or monitor such as the wearable monitor (156).

The first communication by the DCN may include a question such as the question (114) for the individual member. The question may be designed to initiate a conversation between the individual member and the DCN. For example, the question may include “How do you feel about the activity you participated in?”

At Block 208, a step of transmitting the first communication is provided. The first communication may be transmitted to the individual member from the DCN via a user device such as the user device (150). The first communication may be transmitted at a target time determined by the DCN. For example, the first communication may be transmitted after the individual member has completed an activity or an hour after the individual member has woken up. The target time may be a time that the DCN has determined that the individual member is likely to respond to the first communication. The first communication may also be transmitted with, for example, an incentive to the individual member to respond to the first communication. The incentive may be monetary or non-monetary such as a discount, rewards points, or the like.

At Block 210, a step of receiving a first response is provided. The first response may be the same as or similar to the response (112). The first response may be received by the DCN from the individual member via the user device. The first response is received in response to the first communication and may include, for example, an answer to the question. For example, the first communication may be “How do you feel about the activity you participated in?”and the first response may be “I feel good”or “I feel bad”.

At Block 212, a step of analyzing the first response is provided. The first response may be analyzed by, for example, the DCN using the communication analyzer to determine a first level of emotional valence of the individual member. The communication analyzer may search for key words that correspond to different levels of emotional valence. In other embodiments, the communication analyzer may use AI or a machine learning model such as a language model to determine the first level of emotional valence. For example, the first response may be “I feel good” and the communication analyzer may output a high level of emotional valence indicating a positive emotion. In another example, the first response may be “I feel bad” and the communication analyzer may output a low level of emotional valence indicating a negative emotion.

At Block 214, a step of generating a second communication by the DCN is provided. The second communication is designed to induce reflection in the individual member for a first time to increase the first level of emotional valence. For example, if the first emotional valence is low, the second communication may include statements or questions such as “What could you change to improve how you feel?” or “Do you think stretching prior to the activity may help next time?” Such questions may cause the individual member to reflect on how they can improve their emotions or feelings, thereby increasing and improving their level of emotional valence.

At Block 216, a step of transmitting the second communication is provided. The Block 216 may the same as or similar to the Block 208.

At Block 218, a step of receiving a second response is provided. The Block 218 may be the same as or similar to the Block 210.

At Block 220, a step of analyzing the second response is provided. The Block 220 may be the same as or similar to the Block 212 in that the second response may also be analyzed by, for example, the DCN using the communication analyzer to determine a second level of emotional valence of the individual member. After the second level of emotional valence is determined, the method may proceed to either Block 222A or 222B described below. More specifically, if the second level of emotional valence is equal to or less than the first level of emotional valence or if a difference such as the difference (118) between the first level of emotional valence and the second level of emotional valence is equal to or less than an emotional valence threshold such as the emotional valence threshold (120), then the method may proceed to the Block 222A. If the second level of emotional valence is greater than the first level of emotional valence or if the difference is greater than the emotional valence threshold, then the method may proceed to the Block 222B.

At Block 222A, a step of generating a third communication by the DCN is provided. As described above, the third communication may be generated when the second level of emotional valence is equal to or less than the first level of emotional valence or if the difference between the first level of emotional valence and the second level of emotional valence is equal to or less than the emotional valence threshold. The third communication is designed to induce reflection for a second time in the individual member to increase the first level and/or the second level of emotional valence. The third communication may include one or more questions. For example, the third communication may include “Did the change you make improve your activity? If not, why do you think the change did not help? Would a different change help?”. In another example, the first communication may include “How did you feel after your jog?”, the second communication may include “Do you think stretching prior to jogging may help next time?”, and the third communication may include “Do you think eating a snack or being more rested may help next time you jog?”. In still another example, the first communication may include “How did you feel after yoga?”, the second communication may include “Do you think being well rested will improve how you feel during and after yoga next time?”, and the third communication may include “Do you think meditating or reflecting prior to yoga will improve your experience with yoga?”.

The third communication may also be designed to induce reflection by the individual member about a cause of the difference between the first level of emotional valence and the second level of emotional valence or a cause of the second level of emotional valence being less than the first level of emotional valence. For example, the third communication may include “Why do you think you feel worse after yoga?” or “Why do you think you feel worse after jogging?”.

It will be appreciated that the steps 218, 220, and 222A may be repeated when the individual member's emotional valence does not improve or does not improve enough. In other words, subsequent communications (e.g., fourth communication, fifth communication, etc.) may be generated and transmitted and subsequent responses (e.g., third response, fourth response, etc.) may be received and analyzed until the level of emotional valence of the individual member has improved to a positive level.

It will also be appreciated that any of the communications and responses can be sent and received synchronously or asynchronously. Any of the communications and responses can also be sent over any period of time such as, for example, an hour, a day, a week, a month, or several months. Further, any of the communications and responses can be provided verbally and any verbal communications and/or responses can be transcribed into text.

At Block 222B, a step of transmitting a request is provided. As described above, the request may be transmitted when the second level of emotional valence is greater than the first level of emotional valence or if the difference is greater than the emotional valence threshold, indicating a positive shift in the level of emotional valence of the individual member. The request may include a request for the individual member to share their activities and/or reflections in the DCN. The individual member's reflections may encourage other members of the population to try the same activities at the individual member to improve their emotional valence, adverse health condition, or overall well-being. For example, the individual member may share their experience with yoga and/or jogging and how such activities improved their emotional well-being and health. Such experiences may also inspire other members to reflect on their own adverse health conditions and how to improve such health conditions.

The method described in FIG. 2 can include more or less steps. One or more steps or any combination of steps may also be repeated in the method described in FIG. 2.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN) such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302).

Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements: Statement 1. A method to induce reflection, the method comprising: providing a digital communication network (DCN) for members of a population to communicate with each other; analyzing communications in the DCN regarding an individual member, wherein the communications include at least one observation related to a life, lifestyle, or health of the individual member; generating at least one question related to the observations by the individual member based on the analysis; and sending, through communications on the DCN, the at least one question to the individual member to cause the individual member to reflect on the at least one observation.

Statement 2. The method of Statement 1, wherein the at least one observation is made by the individual member in at least one of a post and a message on the DCN.

Statement 3. The method of Statement 1, wherein the at least one observation is in a message by another member of the population which is shared by the individual member.

Statement 4. The method of Statement 1, further comprising deriving the at least one observation from at least one interaction by the individual member using the DCN.

Statement 5. The method of Statement 1, further comprising deriving the at least one observation from biometric data or psychometric data shared with the DCN by the individual member.

Statement 6. The method of Statement 5, wherein the biometric data is at least one of: generated by a wearable device; generated by the individual member engaging in an activity; and supplied by answers to a survey or psychometric instrument.

Statement 7. The method of Statement 6, wherein the biometric data is shared automatically.

Statement 8. The method of Statement 1, further comprising receiving a response to the at least one question by the DCN.

Statement 9. The method of Statement 8, further comprising generating a subsequent question based, at least in part, on the response; and sending, through communications on the DCN, the subsequent question to the individual member.

Statement 10. The method of Statement 1, wherein the at least one question solicits an observation from the individual member.

Statement 11. The method of Statement 1, wherein the at least one question comprises an incentive for the individual member to respond to the at least one question.

Statement 12. The method of Statement 1, wherein the at least one question comprises information relative to the individual member.

Statement 13. The method of Statement 1, wherein the method is further preformed to improve health of the population, reduce health care related costs in the population, and/or slow progression of an adverse health condition in members of the population.

Statement 14. The method of Statement 1, further comprising analyzing communication in the DCN to generate an ontology of health conditions for the individual member, wherein the ontology of health conditions comprises an adverse health condition.

Statement 15. The method of Statement 1, wherein the adverse health condition comprises at least one of: chronic systemic inflammation, malaise, and low energy.

Statement 16. The method of Statement 1, wherein the adverse health condition comprises a disease.

Statement 17. The method of Statement 16, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 18. The method of Statement 1, wherein the adverse health condition comprises a prodromal version of a disease.

Statement 19. The method of Statement 1, wherein the adverse health condition comprises a health risk.

Statement 20. The method of Statement 19, wherein the health risk comprises at least one of: a risk of falling, and a risk of becoming infected.

Statement 21. The method of Statement 1, wherein the adverse health condition comprises social dysfunction.

Statement 22. A method to slow progression of an adverse health condition in a population, the method comprising: providing a digital communication network (DCN) for members of the population to communicate with each other, wherein the DCN comprises a conversational bot; generating a conversation message as part of a conversation with an individual member of the population based on 1) analysis of communications on the DCN, 2) analysis of a response to a prior message in the conversation; and/or 3) analysis of one or more previous conversation with the individual member; and sending, through the DCN, the conversation message to the individual member.

Statement 23. The method of Statement 22, wherein the conversation message is designed to induce reflection by the individual member about an action.

Statement 24. The method of Statement 23, wherein the action is one of: active and passive.

Statement 25. The method of Statement 23, wherein the action is one the individual member undertook or contemplated.

Statement 26. The method of Statement 23, wherein analyzing the communications comprises determining the action.

Statement 27. The method of Statement 26, wherein analyzing the communications comprises analyzing the communications between the individual member and the conversational bot.

Statement 28. The method of Statement 22, wherein the communications comprise at least one of: communications broadly sent on the DCN; and communications from a subset of members on the DCN.

Statement 29. The method of Statement 28, further comprising analyzing content of communications in the DCN and a graph of the DCN to determine the subset of members.

Statement 30. The method of Statement 22, further comprising analyzing communications on the DCN to identify the individual member to have the conversation.

Statement 31. The method of Statement 22, further comprising sending another message providing an incentive for the individual member to respond to the conversation message.

Statement 32. The method of Statement 22, wherein the conversation message directs the individual member to another communication in the DCN.

Statement 33. The method of Statement 32, wherein the another communication includes a reference to an experience that another member of the DCN has engaged in.

Statement 34. The method of Statement 33, wherein the experience is identified as creating happiness or a sense of wellbeing in the another member.

Statement 35. The method of Statement 33, wherein the experience is one of: a lifestyle intervention, a dietary intervention, and a medical intervention.

Statement 36. The method of Statement 35, wherein the experience was unplanned.

Statement 37. The method of Statement 33, wherein the experience is relevant to something the individual member's life, lifestyle, or health.

Statement 38. The method of Statement 33, wherein the experience is relevant to one or more adverse conditions.

Statement 39. The method of Statement 38, further comprising analyzing a plurality of communications in the DCN to determine the one or more adverse conditions, the one or more adverse conditions comprising an adverse condition the individual member is experiencing.

Statement 40. The method of Statement 38, further comprising determining the one or more adverse conditions from a dialog between the individual member and a conversation bot, the one or more adverse conditions comprising an adverse condition the individual member is experiencing.

Statement 41. The method of Statement 22, further comprising presenting communications in the DCN in a prioritized order based on the conversation message.

Statement 42. The method of Statement 22, wherein the conversation message comprises an excerpt of a communication on the DCN.

Statement 43. The method of Statement 42, wherein the excerpt comprises at least one of: post titles, post topics, quotes from the communication, and post contributors.

Statement 44. The method of Statement 42, wherein the excerpt comprises a summaries of communications on the DCN.

Statement 45. The method of Statement 44, wherein the summaries of the communications are created automatically by the DCN.

Statement 46. The method of Statement 44, wherein the summaries of the communications are based on a topic, a set of communications, or a time period.

Statement 47. The method of Statement 22, further comprising presenting a suggestion of a topic for the conversation to the individual member.

Statement 48. The method of Statement 47, wherein the topic is relevant to one or more adverse condition the individual member is experiencing.

Statement 49. The method of Statement 48, further comprising analyzing a plurality of communications in the DCN to determine the one or more adverse conditions, the one or more adverse conditions comprising an adverse condition the individual member is experiencing.

Statement 50. The method of Statement 48, further comprising determining the one or more adverse conditions from a dialog between the individual member and a conversation bot, the one or more adverse conditions comprising an adverse condition the individual member is experiencing.

Statement 51. The method of Statement 22, further comprising generating, by the conversational bot, a post for the individual member to send to the DCN.

Statement 52. The method of Statement 51, wherein the post generated by the conversation bot is formulated in a style designed to provide a positive reaction in the individual member of the population.

Statement 53. The method of Statement 51, wherein the post generated by the conversation bot is formulated in a style designed to provide a positive reaction in members of the population near the individual member in a social graph of the DCN.

Statement 54. The method of Statement 51, wherein the post generated by the conversation bot is formulated in a style designed to provide a positive reaction in members of the population having the adverse health condition.

Statement 55. The method of Statement 22, wherein the conversation with the individual member is held in a virtual reality (VR) environment.

Statement 56. The method of Statement 55, wherein the virtual reality (VR) environment contains a lifestyle, phycological, sociological, or medical intervention.

Statement 57. The method of Statement 55, wherein actions and communications by the individual member as part of the conversation are analyzed in order to generate further communications between the conversation bot and the individual member.

Statement 58. The method of Statement 22, wherein the conversation message directs the individual member to a virtual reality (VR) based environment.

Statement 59. The method of Statement 22, wherein the adverse health condition comprises at least one of: chronic systemic inflammation, malaise, and low energy.

Statement 60. The method of Statement 22, wherein the adverse health condition comprises a disease.

Statement 61. The method of Statement 60, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 62. The method of Statement 22, wherein the adverse health condition comprises a prodromal version of a disease.

Statement 63. The method of Statement 22, wherein the adverse health condition comprises a health risk.

Statement 64. The method of Statement 63, wherein the health risk comprises at least one of: a risk of falling, and a risk of becoming infected.

Statement 65. The method of Statement 22, wherein the adverse health condition comprises social dysfunction.

Statement 66. The method of Statement 22, wherein the conversation message suggests the individual member reflect on a topic.

Statement 67. The method of Statement 22, wherein the conversational bot generates a plurality of additional messages as part of the conversation, wherein the plurality of additional messages focuses on a topic for a period of time.

Statement 68. The method of Statement 67, wherein the period of time is one of: a day, a week, and a month.

Statement 69. The method of Statement 67, wherein the period of time is a number of conversations between the conversation bot and the individual member.

Statement 70. The method of Statement 22, further comprising providing access to tools to enhance reflection on a topic for the individual member.

Statement 71. The method of Statement 70, wherein the tools comprise at least one of: a biosample test, a wearable device, a psychometric instrument, and a structured activity.

Statement 72. The method of Statement 22, wherein the conversational bot generates the conversation message to cause the individual member to reflect on a topic of the conversation.

Statement 73. The method of Statement 22, wherein the conversational bot comprises a plurality of sub-bots, each sub-bot configured to present a specific personality or serve a role.

Statement 74. The method of Statement 22, wherein the method is further preformed to reduce health care related costs in the population, and/or manage a disease in the population.

Statement 75. The method of Statement 22, wherein the method is further preformed to improve the communications of the conversation bot.

Statement 76. A method to induce reflection, the method comprising: providing a digital communication network (DCN) for members of a population to communicate with each other; sending a question to an individual member in the DCN; receiving, in the DCN, an answer to the question; analyzing, by the DCN, the answer to determine an emotional valence of the answer; generating, by the DCN, at least one subsequent question configured to increase the emotional valence of a subsequent response; and sending, through communications on the DCN, the at least one subsequent question to the individual member.

Statement 77. The method of Statement 76, wherein the question, the answer to the question and the at least one subsequent question are sent in an asynchronous communication.

Statement 78. The method of Statement 76, wherein the question, the answer to the question and the at least one subsequent question are sent in a synchronous communication.

Statement 79. The method of Statement 76, wherein the question and the at least one subsequent question are sent over a period of time.

Statement 80. The method of Statement 79, wherein the period of time is one of: an hour, a day, a week, a month, and several months.

Statement 81. The method of Statement 76, wherein the answer is provided verbally.

Statement 82. The method of Statement 81, further comprising automatically transcribing the answer into text.

Statement 83. The method of Statement 76, wherein communications in the DCN comprise at least one of: one-to-one communications, one-to-many communications, one-to-system communications, system-to-one communications, and system-to-many communications.

Statement 84. The method of Statement 76, further comprising analyzing at least one of: communications in the DCN, and biometric test results provided to the DCN, to generate the question.

Statement 85. A method to slow progression of an adverse health condition in a member of a population, the method comprising: providing a digital communication network (DCN) for members of the population to communicate with each other, wherein the DCN comprises a conversational bot; providing access to the conversation bot to an individual member of the population; receiving a message sent to the conversation bot from the individual member as part of a current conversation; generating a reply to the message, wherein the reply is based on 1) analysis of communications on the DCN, 2) analysis of a response to a prior message in the current conversation; and/or 3) analysis of one or more previous conversations with the individual member; and sending the reply to the message through the conversation bot.

Statement 86. The method of Statement 85, wherein the reply is designed to induce reflection by the individual member about an action.

Statement 87. The method of Statement 86, wherein the action is one of: active and passive.

Statement 88. The method of Statement 86, wherein the action is one the individual member undertook or contemplated.

Statement 89. The method of Statement 86, wherein analyzing the communications comprises determining the action.

Statement 90. The method of Statement 89, wherein analyzing the communications comprises analyzing the communications between the individual member and the conversational bot.

Statement 91. The method of Statement 85, wherein the communications comprise at least one of: communications broadly sent on the DCN; and communications from a subset of members on the DCN.

Statement 92. The method of Statement 91, further comprising analyzing content of communications in the DCN and a graph of the DCN to determine the subset of members.

Statement 93. The method of Statement 85, further comprising analyzing communications on the DCN to identify the individual member to have the conversation.

Statement 94. The method of Statement 85, further comprising sending another message providing an incentive for the individual member to respond to the reply.

Statement 95. The method of Statement 85, wherein the reply directs the individual member to another communication in the DCN.

Statement 96. The method of Statement 95, wherein the another communication includes a reference to an experience that another member of the DCN has engaged in.

Statement 97. The method of Statement 96, wherein the experience is identified as creating happiness or a sense of wellbeing in the another member.

Statement 98. The method of Statement 96, wherein the experience is one of: a lifestyle intervention, a dietary intervention, and a medical intervention.

Statement 99. The method of Statement 98, wherein the experience was unplanned.

Statement 100. The method of Statement 96, wherein the experience is relevant to something the individual member is experiencing.

Statement 101. The method of Statement 96, wherein the experience is relevant to one or more adverse conditions.

Statement 102. The method of Statement 101, further comprising analyzing a plurality of communications in the DCN to determine the one or more adverse conditions, the one or more adverse conditions comprising an adverse condition the individual member is experiencing.

Statement 103. The method of Statement 101, further comprising determining the one or more adverse conditions from a dialog between the individual member and a conversation bot, the one or more adverse conditions comprising an adverse condition the individual member is experiencing.

Statement 104. The method of Statement 85, further comprising presenting communications in the DCN in a prioritized order based on the conversation message.

Statement 105. The method of Statement 85, wherein the reply comprises an excerpt of a communication on the DCN.

Statement 106. The method of Statement 105, wherein the excerpt comprises at least one of: post titles, post topics, quotes from the communication, and post contributors.

Statement 107. The method of Statement 105, wherein the excerpt comprises a summaries of communications on the DCN.

Statement 108. The method of Statement 107, wherein the summaries of the communications are created automatically by the DCN.

Statement 109. The method of Statement 107, wherein the summaries of the communications are based on a topic, a set of communications, or a time period.

Statement 110. The method of Statement 85, further comprising presenting a suggestion of a topic for the conversation to the individual member.

Statement 111. The method of Statement 110, wherein the topic is relevant to one or more adverse condition the individual member is experiencing.

Statement 112. The method of Statement 111, further comprising analyzing a plurality of communications in the DCN to determine the one or more adverse conditions, the one or more adverse conditions comprising an adverse condition the individual member is experiencing.

Statement 113. The method of Statement 111, further comprising determining the one or more adverse conditions from a dialog between the individual member and a conversation bot, the one or more adverse conditions comprising an adverse condition the individual member is experiencing.

Statement 114. The method of Statement 85, further comprising generating, by the conversational bot, a post for the individual member to send to the DCN.

Statement 115. The method of Statement 114, wherein the post generated by the conversation bot mimics a style of the individual member.

Statement 116. The method of Statement 114, wherein the post generated by the conversation bot is formulated in a style designed to provide a positive reaction in members of the population near the individual member in a social graph of the DCN.

Statement 117. The method of Statement 114, wherein the post generated by the conversation bot is formulated in a style designed to provide a positive reaction in members of the population having the adverse health condition.

Statement 118. The method of Statement 85, wherein the conversation with the individual member is held in a virtual reality (VR) environment.

Statement 119. The method of Statement 118, wherein the virtual reality (VR) environment contains a lifestyle, phycological, sociological, or medical intervention.

Statement 120. The method of Statement 118, wherein actions and communications by the individual member as part of the conversation are analyzed in order to generate further communications between the conversation bot and the individual member.

Statement 121. The method of Statement 85, wherein the reply directs the individual member to a virtual reality (VR) based environment.

Statement 122. The method of Statement 85, wherein the adverse health condition comprises at least one of: chronic systemic inflammation, malaise, and low energy.

Statement 123. The method of Statement 85, wherein the adverse health condition comprises a disease.

Statement 124. The method of Statement 123, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 125. The method of Statement 124, wherein the adverse health condition comprises a prodromal version of a disease.

Statement 126. The method of Statement 124, wherein the adverse health condition comprises a health risk.

Statement 127. The method of Statement 126, wherein the health risk comprises at least one of: a risk of falling, and a risk of becoming infected.

Statement 128. The method of Statement 85, wherein the adverse health condition comprises social dysfunction.

Statement 129. The method of Statement 85, wherein the reply suggests the individual member reflect on a topic.

Statement 130. The method of Statement 85, wherein the conversational bot generates a plurality of additional messages as part of the conversation, wherein the plurality of additional messages focuses on a topic for a period of time.

Statement 131. The method of Statement 130, wherein the period of time is one of: a day, a week, and a month.

Statement 132. The method of Statement 130, wherein the period of time is a number of conversations between the conversation bot and the individual member.

Statement 133. The method of Statement 85, further comprising providing access to tools to enhance reflection on a topic for the individual member.

Statement 134. The method of Statement 133, wherein the tools comprise at least one of: a biosample test, a wearable device, a psychometric instrument, and a structured activity.

Statement 135. The method of Statement 85, wherein the conversational bot generates the reply to cause the individual member to reflect on a topic of the conversation.

Statement 136. The method of Statement 85, wherein the conversational bot comprises a plurality of sub-bots, each sub-bot configured to present a specific personality or serve a role.

Statement 137. The method of Statement 85, wherein the method is further preformed to reduce health care related costs in the population, and/or manage a disease in the population.

Statement 138. The method of Statement 85, wherein the method is further preformed to improve the communications of the conversation bot.

What is claimed is:

1. A method to induce reflection, the method comprising:
providing a digital communications network (DCN) for a plurality of members of a population to communicate with each other;
transmitting a first communication by the DCN to an individual member of the plurality of members;
receiving a first response to the first communication from the individual member;
analyzing the first response to determine a first level of emotional valence of the individual member;
generating a second communication by the DCN designed to induce reflection in the individual member for a first time to increase the first level of emotional valence;
transmitting the second communication to the individual member;
receiving a second response to the second communication from the individual member; and
analyzing the second response to determine a second level of emotional valence of the individual member.

2. The method of claim 1, further comprising:
generating a third communication by the DCN designed to induce reflection for a second time in the individual member to increase the first level of emotional valence when the second level of emotional valence is less than the first level of emotional valence.

3. The method of claim 1, further comprising:
generating a third communication by the DCN designed to induce reflection for a second time in the individual member to increase the first level of emotional valence when a difference between the first level of emotional valence and the second level of emotional valence is less than an emotional valence threshold.

4. The method of claim 1, further comprising:
transmitting a request to the individual member to share their reflection in the DCN when a difference between the first level of emotional valence and the second level of emotional valence is greater than an emotional valence threshold.

5. The method of claim 1, further comprising:
analyzing at least one of: one or more communications in the DCN by the individual member or biometric test results provided to the DCN by the individual member to generate the first communication and the second communication.

6. The method of claim 1, wherein the first communication and the second communication comprise a first question and a second question, respectively.

7. The method of claim 1, wherein transmitting the first communication includes transmitting an incentive with the first communication.

8. The method of claim 1, wherein the induced reflection in the second communication is designed to induce reflection by the individual member about an action the individual member took.

9. The method of claim 3, wherein the induced reflection in the third communication is designed to induce reflection by the individual member about a cause of the difference between the first level of emotional valence and the second level of emotional valence.

10. The method of claim 2, wherein the induced reflection in the third communication is designed to induce reflection by the individual member about a cause of the second level of emotional valence being less than the first level of emotional valence.

11. A method to induce reflection, the method comprising:
providing a digital communications network (DCN) for a plurality of members of a population to communicate with each other;
transmitting a first communication to an individual member of the plurality of members;
receiving a first response to the first communication;
analyzing the first response to determine a first level of emotional valence of the individual member;
generating a second communication designed to induce reflection in the individual member for a first time to increase the first level of emotional valence;
transmitting the second communication to the individual member;
receiving a second response to the second communication;
analyzing the second response to determine a second level of emotional valence of the individual member; and
generating a third communication designed to induce reflection for a second time in the individual member to increase the first level of emotional valence when a difference between the first level of emotional valence and the second level of emotional valence is less than an emotional valence threshold.

12. The method of claim 11, wherein the third communication by the DCN is generated when the second level of emotional valence is less than the first level of emotional valence.

13. The method of claim 11, further comprising:
transmitting a request to the individual member to share their reflection in the DCN when a difference between the first level of emotional valence and the second level of emotional valence is greater than an emotional valence threshold.

14. The method of claim 11, further comprising:
analyzing at least one of: one or more communications in the DCN by the individual member or biometric test results provided to the DCN by the individual member to generate the first communication and the second communication.

15. The method of claim 11, wherein the first communication and the second communication comprise a first question and a second question, respectively.

16. The method of claim 11, wherein transmitting the first communication includes transmitting an incentive with the first communication.

17. The method of claim 11, wherein the induced reflection in the second communication is designed to induce reflection by the individual member about an action the individual member took.

18. The method of claim 11, wherein the induced reflection in the third communication is designed to induce reflection by the individual member about a cause of the difference between the first level of emotional valence and the second level of emotional valence.

19. The method of claim 12, wherein the induced reflection in the third communication is designed to induce reflection by the individual member about a cause of the second level of emotional valence being less than the first level of emotional valence.

20. A system to induce reflection, the system comprising:
a computer processor;
a data repository in communication with the computer processor and storing:
a plurality of communications including a first communication and a second communication;
a plurality of responses including a first response and a second response; and
a plurality of levels of emotional valence including a first level of emotional valence and a second level of emotional valence;
a digital communications network (DCN) which, when executed by the computer processor, provides a network for a plurality of members of a population to interact with each other; and
a server controller which, when executed by the computer processor:
transmits the first communication from the DCN to an individual member of the plurality of members;
receives the first response to the first communication from the individual member;
analyzes the first response to determine the first level of emotional valence of the individual member;
generates the second communication designed to induce reflection in the individual member for a first time to increase the first level of emotional valence;
transmits the second communication to the individual member from the DCN;
receives a second response to the second communication from the individual member; and
analyzes the second response to determine the second level of the emotional valence.
